(19) United States
(12) Patent Application Publication
(10) Pub. No.: US 2011/0245623 A1
(43) Pub. Date: Oct. 6, 2011

(54) Medical Diagnosis Using Community Information

(75) Inventors: Sailesh Chutani, Redmond, WA (US); David M. Zar, Maryland Heights, MO (US); Nikhil J. George, Redmond, WA (US)

(73) Assignee: MobiSante Inc., Redmond, WA (US)

(21) Appl. No.: 12/938,333

(22) Filed: Nov. 2, 2010

Related U.S. Application Data
(60) Provisional application No. 61/400,709, filed on Aug. 2, 2010.
(60) Provisional application No. 61/341,734, filed on Apr. 5, 2010.

(51) Int. Cl. A61B 5/00

(52) U.S. Cl. 600/300

(57) ABSTRACT

A computer includes one or more processors; and logic coupled to the one or more processors and comprising one or more stored sequences of instructions which, when executed by one or more processors, cause the one or more processors to perform: collecting first patient data, associated with at least one attribute, from a patient by one or more biometric sensors based on a first protocol; evaluating the first patient data based at least on a portion, of aggregate medical information, associated with the at least one attribute; based on the evaluating, determining a second protocol for collecting additional patient data; collecting the additional patient data by the one or more biometric sensors based on the second protocol.

### CROSS-REFERENCE TO RELATED APPLICATIONS; BENEFIT CLAIM

This application claims the benefit under 35 U.S.C. §119(e) of Provisional Appln. 61/400,709, filed Aug. 2, 2010, the entire content of which is hereby incorporated by reference as if fully set forth herein.

This application claims the benefit under 35 U.S.C. §119(e) of Provisional Appln. 61/341,734, filed Apr. 5, 2010, the entire content of which is hereby incorporated by reference as if fully set forth herein.

### TECHNICAL FIELD

The present disclosure generally relates to medical diagnosis using community information. The disclosure relates more specifically to determining a protocol for a medical diagnosis based on aggregate medical information related to a patient community.

### BACKGROUND

The approaches described in this section could be pursued, but are not necessarily approaches that have been previously conceived or pursued. Therefore, unless otherwise indicated herein, the approaches described in this section are not prior art to the claims in this application and are not admitted to be prior art by inclusion in this section.

The advent of medical diagnostic devices has changed the manner in which medical personnel collect and evaluate patient data. Medical diagnostic devices include biometric sensors such as ultrasound probes which can collect patient data for visualizing subcutaneous body structures including tendons, muscles, joints, vessels and internal organs for possible pathology or lesions. For example, obstetric sonography, which is commonly used during pregnancy may be used to visualize a fetus.

Traditionally medical diagnostic devices have been large in size and stationed in particular rooms within a hospital setting or medical office. Recently, portable medical diagnosis devices have been developed for collecting data from patients in their homes, medical offices, or other suitable locations. The portable medical diagnosis devices are generally lower in costs and are more accessible for patients.

### BRIEF DESCRIPTION OF THE DRAWINGS

In the drawings:

FIG. 1A illustrates a computer system in accordance with an embodiment;

FIG. 1B illustrates an example of biometric sensor management logic;

FIG. 2 illustrates determining a protocol;

FIG. 3 illustrates an example of a biometric sensor;

FIG. 4 and FIG. 5 illustrate examples of one or more computers upon which one or more embodiments may be implemented.

### DETAILED DESCRIPTION OF ONE OR MORE EXAMPLE EMBODIMENTS

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

### General Overview

In an embodiment, a method includes: collecting first patient data, associated with at least one attribute, from a patient by one or more biometric sensors based on a first protocol; evaluating, by a computer, the first patient data based at least on a portion, of aggregate medical information, associated with the at least one attribute; based on the evaluating, determining a second protocol for collecting additional patient data; collecting the additional patient data by the one or more biometric sensors based on the second protocol.

Evaluating the first patient data based at least on the portion of the aggregate medical information may include: determining that the first patient data comprises one or more indications of a particular medical diagnosis; wherein the second protocol tests for the particular medical diagnosis.

Evaluating the first patient data based at least on the portion of the aggregate medical information may include: computing a confidence measure that the first patient data is indicative of a particular medical diagnosis; wherein the confidence measure is based, at least in part, on a frequency of the particular medical diagnosis in patients associated with the portion of the aggregate medical information.

In an embodiment, the method includes: prior to obtaining the first patient data by the one or more biometric sensors, obtaining the aggregate medical information by the one or more biometric sensors and tagging the portion of the aggregate medical information with the at least one attribute.

In an embodiment, the method includes: detecting that the first patient data was collected at a particular location with a geographical area; determining that the portion of the aggregate medical information was collected within the geographical area; responsive to the determining that the portion of the aggregate medical information was collected within the geographical area, selecting the portion of the aggregate medical information for evaluating the first patient data.

In an embodiment, an attributes may include one or more of: genetic information of the patient; an age of the patient; a nationality of the patient; an ethnicity of the patient; a place of residence of the patient; a place of work of the patient; a socio-economic group associated with the patient; a behavioral habit of the patient; a lifestyle habit of the patient; a patient condition in which the first patient data was collected; an environmental condition to which the patient is exposed; a chemical to which the patient is exposed; or a pollutant to which the patient is exposed.

In an embodiment, the computer may be communicatively coupled, directly, with the one or more biometric sensors.

In an embodiment, the method includes: generating a geographical map comprising a first plurality of locations at which the portion of the aggregate medical information was collected and a second plurality of locations associated with environmental factors.

The first patient data may include one or more results of an ultrasound probing procedure performed with an ultrasound probe comprising the one or more biometric sensors.

The first patient data and the additional patient data may be collected by the one or more biometric sensors within a same medical examination session.

In an embodiment, a method includes: obtaining, from a remote computer, first patient data associated with at least one attribute and collected from a patient by one or more biometric sensors based on a first protocol; evaluating the first patient data based at least on a portion, of aggregate medical information, associated with the at least one attribute; based on the evaluating, determining a second protocol for collecting additional patient data; obtaining, from the remote computer, the additional patient data collected by the one or more biometric sensors based on the second protocol within a same medical examination session as the first patient data.

The portion of the aggregate medical information may be collected within a particular geographical area and the method may further include: determining that the remote computer is located within the particular geographical area; responsive to determining that the remote computer is located within the particular geographical area, selecting the portion of the aggregate medical information collected within the particular geographical area.

In an embodiment, a method includes: storing patient data collected by one or more biometric sensors as aggregate medical information; tagging one or more portions of the aggregate medical information with one or more attributes; determining that particular patient data, that is to be collected by the one or more biometric sensors, is associated with at least one attribute of the one or more attributes; responsive to determining that the particular patient data is associated with at least one attribute, selecting a portion of the aggregate medical information that is tagged with the at least one attribute; determining a common medical diagnosis associated with the portion of the aggregate medical information; identifying a protocol for testing a patient for the common medical diagnosis; based on the protocol, collecting the particular patient data from the patient by the one or more biometric sensors; wherein the method is performed by at least one computer.

In other aspects, the disclosure encompasses an apparatus with means for the functionality described herein and a computer readable medium comprising instructions, which when executed by one or more processors provide the functionality described herein.

In other aspects, the disclosure encompasses at least one computer performing one or more method steps as described herein.

### Structural Overview

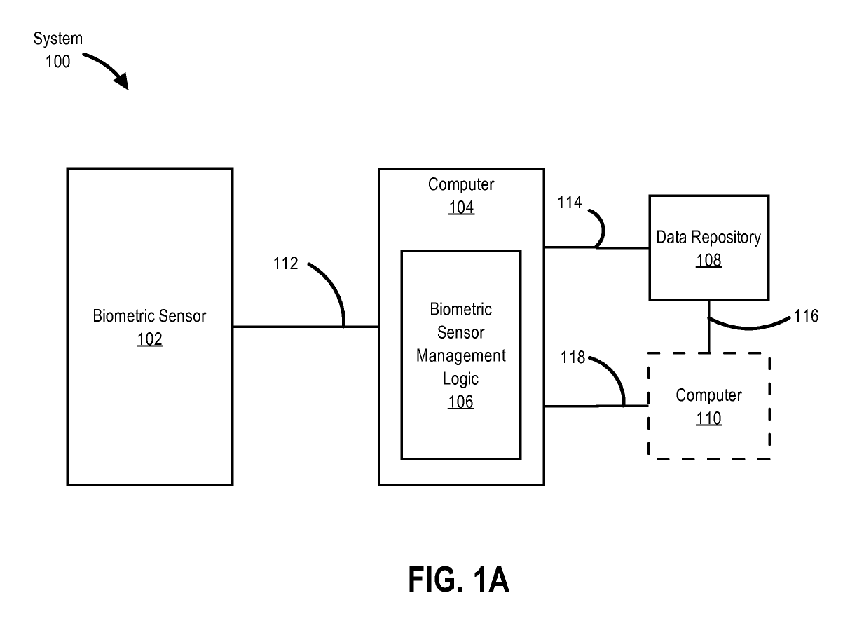

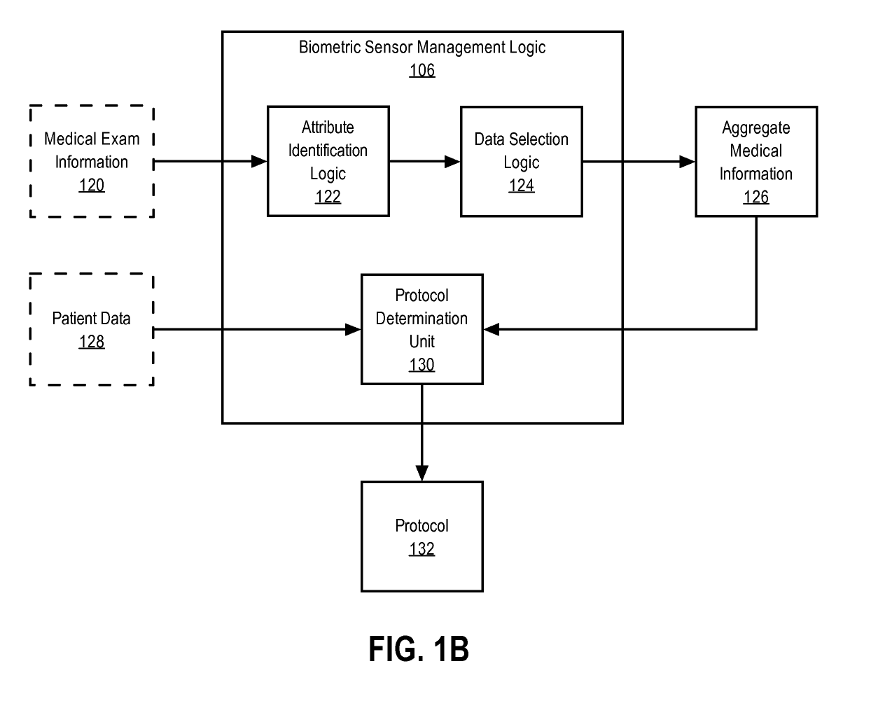

FIG. 1A illustrates a system in accordance with an embodiment. Although a specific system is described, other embodiments are applicable to any system that can be used to perform the functionality described herein. FIG. 1A illustrates a hypothetical system 100. Components of the system 100 may be connected by, without limitation, a network such as a Local Area Network (LAN), Wide Area Network (WAN), the Internet, Intranet, Extranet with terrestrial, satellite or wireless links, etc. Thus, in an embodiment, links 112, 114, 116, and 118 may each comprise a network link or cable. Alternatively or additionally, any number of devices connected within the network may also be directly connected to each other through wired or wireless communication segments. One or more components described within system 100 may be combined together in a single device. For example, sensor 102 may be integrated with computer 104; and computer 110 and data repository 108 may be remotely coupled with computer 104 through one or more networks.

In an embodiment, the system 100 includes one or more biometric sensors (e.g., biometric sensor 102), one or more computers (e.g., computer 104 and computer 110), and one or more data repositories (e.g., data repository 108).

In an embodiment, the biometric sensor 102 generally represents any sensor used to collect data related to a patient, which may be referred to herein as patient data. Patient data may include, without limitation, raw data collected from a patient, an analysis of the patient data, textual information based on raw data, or images based on the raw data. The biometric sensor 102 may collect patient data, for example, within a particular range from the patient, in direct contact with the patient, or applied to the patient through a conductive medium (e.g., gel).

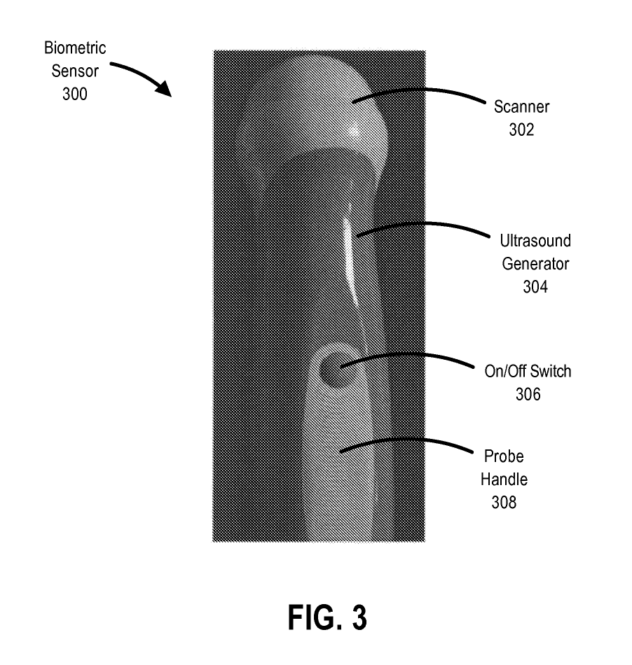

A biometric sensor 102 may refer to, for example, an ultrasound probe which collects patient data through sound waves (e.g., with a frequency of 3.5 MHz, 5 MHz, 7.5 MHz, 12 MHz, etc.). FIG. 3 illustrates an ultrasound probe 300 as an example of a biometric sensor 102. An ultrasound probe may include a scanner 302, an ultrasound generator 304 to generate sound waves that are applied toward a patient through a gel or other conductive medium, an on/off switch 306, and a probe handle 308. An ultrasound probe may further include a receiver for capturing sound wave echoes which are used to generate image data to visualize subcutaneous body structures (e.g., tendons, muscles, joints, vessels, internal organs, fetuses in pregnant women). A biometric sensor 102 may be a handheld device which is operated by an operator (e.g., human or robotic operator). Other examples of biometric sensors include, without limitation, medical cameras, electrocardiogram sensors, pulse oxymeters, and blood glucose monitors.

In an embodiment, the biometric sensor 102 may be used to collect patient data based on a protocol. A protocol generally represents directions for any procedure performed by an operator of the biometric sensor 102. A protocol may be represented in data stored in computer storage. A protocol may define organs that are to be probed and/or measured, actions that are to be performed by an operator, biometric sensor settings (e.g., gain control, intensity, contrast, depth, etc.), locations on a patient where the biometric sensor 102 is to be placed, etc.

In an embodiment, each protocol may correspond to one or more exams. For example, a protocol may define a particular procedure to test for symptoms or indications related to a particular disease or other medical diagnosis.

Furthermore, protocols may differ based on the patient. For example, thin patients may require a different protocol than obese patients in order to obtain useful patient data.

In an embodiment, computer 104 generally represents any device that includes a processor and is communicatively coupled with the biometric sensor 102. Examples of computer 104 include, without limitation, a desktop, a laptop, a tablet, a cellular phone, a smart phone, a personal digital assistant, a kiosk, etc. Computer 104 may be communicatively coupled with the biometric sensor 102 with wired and/or wireless segments. Computer 104 may be connected directly with the biometric sensor 102 using a universal serial bus (USB) cable.

Computer 104 may be used for determining or receiving one or more protocols for use with the biometric sensor 102 to collect patient data as described below with respect to FIG. 1B. In an embodiment, computer 104 includes a biometric sensor management logic 106, which may comprise firmware, hardware, software, or a combination thereof in various embodiments that can implement the functions described herein.

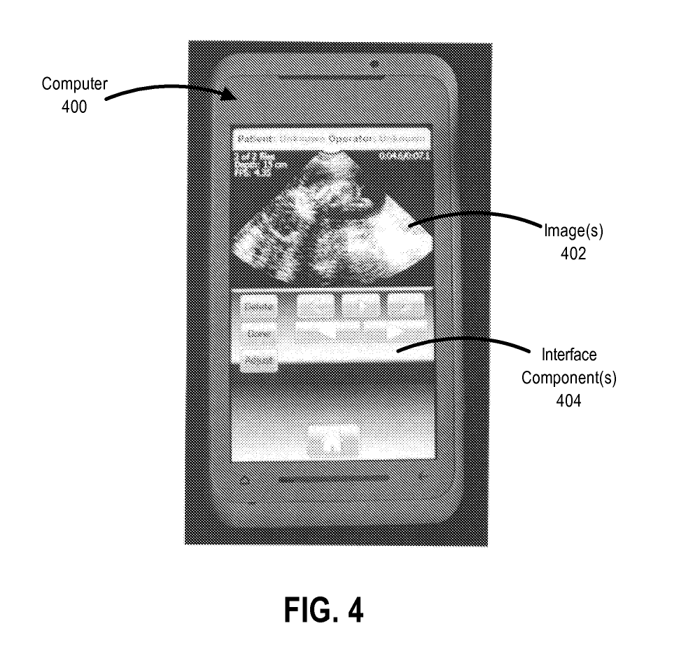

FIG. 4 illustrates a computer 400, as an example of computer 104 that may be used with a biometric sensor 102 such as an ultrasound probe. In an embodiment, computer 400 may include one or more buffers for temporarily or permanently recording patient data. For example, computer 400 may include logic configured to display images 402 (or any other patient information) based on the patient data collected by the biometric sensor 102. Data recorded in any buffer within computer 400 may be sampled at varying rates and using varying techniques. For example, every other image within a buffer may be sampled and transmitted to another computer (e.g., computer 110). In another example, every other horizontal vector or vertical vector from each image may be sampled and transmitted. A portion of interest of each image may be selected and transmitted. Different buffers within computer 400 may record the same patient data with varying levels of quality. For example, a particular buffer may include all patient data and another buffer may include a portion (e.g., based on sampling rate) of the patient data.

In an embodiment, the computer 400 may be configured to tag patient data with one or more attributes. Examples of attributes include, without limitation, patient information, geographical information, and environmental information. Patient information may refer to nationality, ethnicity, place of residence, place of work, socio-economic group, age, genetic characteristics, behavioral habits, lifestyle habits, etc. of a patient from whom the patient data is collected. Patient information may further include a patient condition when the patient data was collected. For example, the patient information may indicate that the patient data was collected after a twelve hour fast, after a five minute jog, etc. Geographical information may refer to a location where the patient data was collected, information about a location where the patient data was collected, etc. Environmental information may refer to specific chemicals (e.g., pollutants found in land, water, or air within a geographical area) within an environment, general environmental quality ratings or conditions, etc. In an embodiment, the computer 400 may be configured to categorize patient data based on one or more attributes. For example, the computer 400 may be configured to manage, access, edit, or search patient data using a query for patients over the age of sixty-five that smoke tobacco. In an embodiment, patient data collected by one or more biometric sensors 102 may be stored on a local computer 104 or a remote computer 110 as aggregate medical information 126, described below in relation to FIG. 1B.

In an embodiment, computer 400 may include one or more interface components 404 to add, edit, delete, display, or send data accessible to the computer 400. The interface components 404 may be used to manage the biometric sensor 102.

In an embodiment, the data repository 108 generally represents any data storage device known in the art which may be configured to store data. Examples include local memory on computer 104, local memory on computer 110, shared memory, multiple servers connected over the internet, systems within a local area network, a memory on a mobile device, etc. In one or more embodiments, access to the data repository 108 may be restricted and/or secured. Access to the data repository 108 may require authentication using passwords, secret questions, personal identification numbers (PINs), and/or any other suitable authentication mechanism. Portions of data stored in the data repository 108 may be distributed and stored in multiple data repositories (e.g., servers across the world).

In one or more embodiments, the data repository 108 includes flat, hierarchical, network based, relational, dimensional, object modeled, or data files structured otherwise. For example, data repository 108 may be maintained as a table of an SQL database. In addition, data in the data repository 108 may be verified against data stored in other repositories.

Computer 110 may be implemented as described herein in relation to computer 104. Computer 110 may be located remotely from biometric sensor 102 and computer 104. Computer 110 may obtain data collected by the biometric sensor 108 directly from the biometric sensor 108 or via computer 104. Computer 110 may be operated by a remote user to provide instructions which are transmitted to computer 104. Computer 104 or computer 110 may be configured to determine or receive one or more protocols for operating the biometric sensor 108. Computer 104 or computer 110 may comprise an analysis workstation for evaluating patient data.

### Architectural and Functional Overview

FIG. 1B illustrates an example of a biometric sensor management logic 106 located on computer 104. In an embodiment, the biometric sensor management logic 106 comprises an attribute identification logic 122, a data selection logic 124, and a protocol determination unit 130. One or more components of the biometric sensor management logic 106 may be located on a different computer (e.g., computer 110) that is communicatively coupled with computer 104.

In an embodiment, the attribute identification logic 122 may comprise hardware, firmware, and software to determine one or more attributes associated with patient data that is already collected or patient data that will be collected. The one or more attributes determined by the attribute identification logic 122 may be detected or received as input by a user or computer. The attribute identification logic 122 may use any medical exam information 120 such as information related to an exam type, the environment where the exam is to be performed, the patient(s) on whom the exam is to be administered, etc.

For example, the attribute identification logic 122 may be configured to receive or determine a current location of computer 104. The attribute identification logic 122 may comprise global positioning system (GPS) electronics to receive GPS signals from GPS satellites. Based on the GPS signals, the attribute identification logic 122 may determine the location of computer 104. The attribute identification logic 122 may also determine the location of computer 104 based on signals received from cellular phone towers. The attribute identification logic 122 may also obtain the current location from user input. The attribute identification logic 122 may be configured to receive or determine a future location of computer 104 where patient data 124 is to be collected. The location identified by the attribute identification logic 122 may be an exact location, a point of interest, or a general area (e.g., village, town, city, state, etc.). Locations, as referred to herein, may refer to regions affected by particular environmental factors (e.g., high elevation location, high pollution location) or related to particular medical diagnoses (e.g., high diabetes rate area, high cholesterol rate area).

In an embodiment, the attribute identification logic 122 may be configured to analyze a blood sample, hair sample, fingerprint, urine sample, saliva sample, or other sample obtained from a patient to identify an attribute. For example, the attribute identification logic 122 may analyze a hair sample to extract deoxyribonucleic acid (DNA) and determine genetic information based on the DNA.

In an embodiment, the data selection logic 124 may comprise hardware, firmware, and software to select aggregate medical information 126 based on one or more attributes associated with patient data. The aggregate medical information 126 generally represents a collection of medical information for a group of patients that is selected based on one or more attributes. For example, the aggregate medical information 126 may be associated with patients in a particular geographical area which includes a location identified by the attribute identification logic 122. If a particular address is identified, the aggregate medical information 126 may be associated with patients within a city or town that includes that particular address.

The aggregate medical information 126 may be associated with patients in similar regions as the identified location. For example, if the identified location is in a highly polluted area, the aggregate medical information may be associated with patients in geographical areas that are highly polluted.

In an embodiment, the aggregate medical information 126 may be associated with patients with a common attribute (e.g., nationality, ethnicity, place of residence, socio-economic groups, age, genetic characteristics, behavioral habits, lifestyle habits, etc.). The aggregate medical information 126 may include any patient data, analysis of patient data, or trends associated with patient data. For example, the aggregate medical information 126 may include one or more of: raw patient data, common symptoms, or a common medical diagnosis for patients associated with one or more common attributes. The aggregate medical information 126 may be textual or graphical (e.g., still images, videos, etc.). The aggregate medical information 126 may include patient data with or without corresponding patient identification information.

In an embodiment, the protocol determination unit 130 is configured to determine (includes selecting) one or more protocols (e.g., protocol 132) for collecting patient data with the biometric sensor 102. As described above, a protocol generally represents any directions for a procedure performed by an operator of the biometric sensor 102 for collecting patient data via the biometric sensor 102. In an embodiment, the protocol determination unit 130 may determine the protocol 132 based on the aggregate medical information 126 and/or the patient data 128. The protocol determination unit 130 may continuously, periodically, upon receiving data, or upon user request, update the protocol 132. For example, based on patient data which includes one or more minor indications of a particular disease, the protocol determination unit 130 may update the protocol 132 to test for that particular disease.

All components of the biometric sensor management logic 106 may be integrated into a single unit of software, firmware, or a combination. Thus, the separate blocks shown in FIG. 1B are provided solely to illustrate one example.

### Protocol Determination

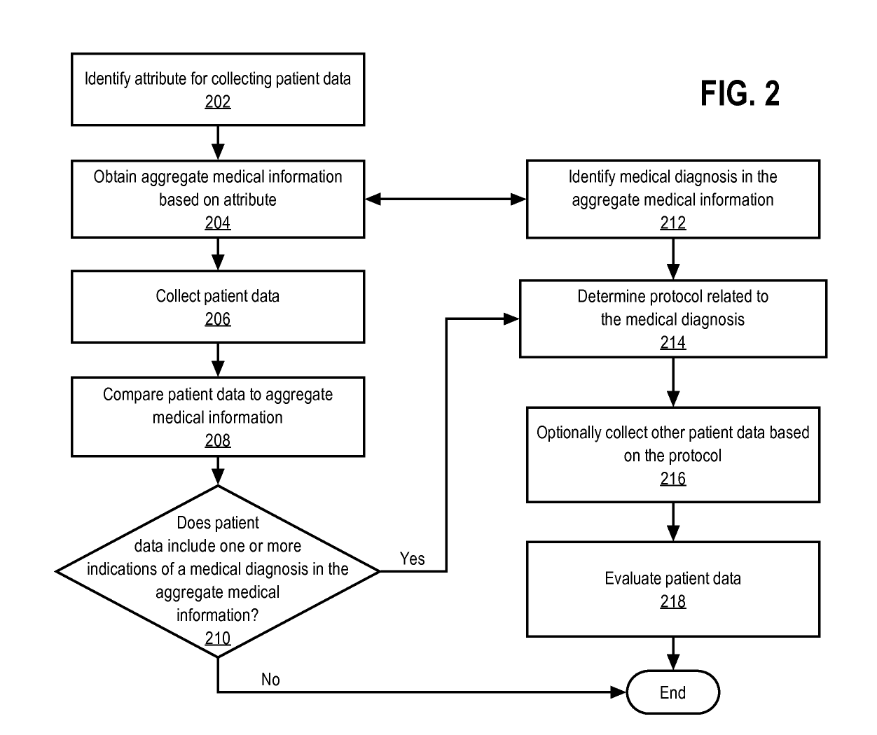

FIG. 2 illustrates determining a protocol for use with one or more biometric sensors. FIG. 2 may represent an algorithm that may be embodied in or hosted by the protocol determining unit 130. In an embodiment, one or more of the steps described below may be omitted, repeated, or performed in a different order. The specific arrangement shown in FIG. 2 is not required.

In Step 202, an attribute associated with patient data that will be collected is identified. The attribute may include a location that is identified based on a current location of a device that is to be used for collecting the patient data. For example, a user may enter a command to identify the current location. In response to the command, a device may analyze satellite signals that are being received and determine the current location based on the satellite signals. In an embodiment, the location may be determined based on a location schedule. For example, scheduled patient appointments may be queried with a particular date and time to determine a location of the appointment corresponding to the date and time. Identifying a location may include prompting a user to enter the location or obtaining location data from stored configuration data.

In an embodiment, an attribute may be detected based on a blood sample, hair sample, fingerprint, urine sample, saliva sample, or other patient sample obtained from a patient. For example, an analysis of a blood sample may be used to detect traces of heroine and identify “drug user” as an attribute. In another example, a body mass index (BMI) may be measured to identify “obese” as an attribute.

In Step 204, aggregate medical information may be obtained (or selected) based on the attribute. The aggregate medical information may be collected by the one or more biometric sensor and stored locally at computer 104 connected to biometric sensor 102. The aggregate medical information may be obtained by querying a database with the attribute. For example, a database may be queried with a location or with a geographical area that includes the location for a medical examination. The aggregate medical information may be obtained by querying a database with characteristics associated with the location. For example, if Linfen, China was identified as the location, the database may be queried with “high coal pollution area.” In an embodiment, one or more portions of the aggregate medical information may be requested. For example, raw patient data or common medical diagnoses may be specifically requested and obtained. In an embodiment, a database may be queried for symptoms of common medical diagnoses for a particular patient group (e.g., a patient group within a specific geographical area).

In an embodiment, the aggregate medical information may be filtered based on a time range. For example, in a city exposed to radiation for the last six months, aggregate medical information for patients over sixty-five with cancer may be filtered to include patient data that was collected within the last six months. This filtered aggregate medical information may be tailored to identify the effects of particular environmental circumstances.

In Step 206, patient data is collected. In an embodiment, patient data may be collected by one or more biometric sensors which are operated by a human or machine operator. Collecting the patient data may involve an operator placing a biometric sensor such that the biometric sensor can collect the patient data and cause storing of the patient data in a computer that is communicatively coupled to the biometric sensor. For example, a biometric sensor may be placed within a particular range from a patient, may be placed in direct contact with the patient's skin, may be inserted inside the patient (e.g., inside a patient's mouth), may be placed in indirect contact with the patient's skin (e.g., contacting a conductive gel that is on the patient), etc.

Collecting patient data may include following one or more protocols. For example, collecting patient data may include configuring one or more settings (software or hardware) for the biometric sensor, moving the biometric sensor in a particular direction, and moving the biometric sensor with a particular speed or acceleration based on a protocol. Collecting patient data may include attaching or removing one or more components from the biometric sensor based on a protocol. In an embodiment, collecting patient data may include automatically configuring settings in a biometric sensor by a computer that obtains information related to an exam being performed.

Collecting patient data may include configuring a computer attached to the biometric sensor. For example, a computer used concurrently with the biometric sensor to receive, store, review, or analyze patient data collected by the biometric sensor may be configured to display the patient data in a particular format. The computer may be configured to filter out portions of the patient data before display of the patient data.

In an embodiment, the computer may be configured to obtain data at particular time intervals or frequencies. The computer may be configured to execute instructions stored on a computer readable storage medium that are applicable to the protocol. For example, different display modes or different software may be selected based on the protocol. In an embodiment, collecting patient data may include configuring a first computer that receives the patient data, to immediately transmit the patient data to a different computer. For example, with reference to FIG. 1A, computer 104 may obtain patient data from the biometric sensor 102 and stream the patient data (or a sample of the patient data) to computer 110 over one or more networks as the patient data is being received. Streaming the patient data may include streaming images or other patient information that is generated from the raw data collected by the biometric sensor 102.

In Step 208, patient data is evaluated at least by comparing the patient data to the aggregate medical information that is based on the location identified in Step 202. Comparing the patient data may include a computer-based comparison of symptoms of a current patient with symptoms related to a common medical diagnosis in the aggregate medical information conduct at either of computer 104 or computer 110. If the symptoms of a current patient match the symptoms related to a common medical diagnosis, then the current patient may be tested specifically for that common medical diagnosis. In an embodiment, a score that is indicative of a likelihood or confidence measure that a patient suffers from a particular medical condition may be determined based on the patient data. The score may be increased or decreased based on the aggregate medical information associated with patients in the same patient community (e.g., sharing one or more attributes with the patient). For example, if a patient community that includes a patient is prone to a specific medical condition, then a confidence measure of the patient suffering from the same medical condition may be increased. Conversely, if the specific medical condition is not common in the patient community, the confidence measure of the patient suffering from the same medical condition may be decreased.

In an embodiment, a common medical diagnosis may refer to a medical diagnosis that is found in a threshold number or threshold percentage of the patient population corresponding to the aggregate medical information. In an embodiment, a common medical diagnosis may refer to a medical diagnosis that is found with increasing frequency over time in the patient population corresponding to the aggregate medical information. In an embodiment, the comparison of the patient data with the aggregate medical information may be performed by computer 104 or computer 110, which may identify one medical diagnosis that the current patient should be tested for.

In an embodiment, one or more common medical diagnosis found within the aggregate medical information may be identified without evaluating the patient data of the current patient, as shown in Step 212. For example, in a particular patient community comprising of patients that are exposed to high levels of asbestos, all patients may be tested for a common medical condition of asbestosis which is caused by asbestos.

In Step 214, a protocol related to the common medical diagnosis identified in the aggregate medical information is determined. Determining the protocol may be performed by computer 104 or computer 110 querying a database 108 with the common medical diagnosis and searching for a corresponding protocol to test for that common medical diagnosis. Determining the protocol may involve transmitting the common medical diagnosis to a remote system and receiving the protocol from the remote system.

In an embodiment, the protocol may be loaded onto a computer communicatively coupled to a biometric sensor, in response to detecting that a location. For example, if a set of protocols are commonly used within a particular geographical area, a computer may automatically be updated with the set of protocols in response to determining that the computer is within the particular geographical area. In an embodiment, the protocol may be selected from a set of protocols that is applicable for the particular geographical area. For example, protocol options that are presented to a user may be dynamically modified based on a location where the patient data is being collected. A user may then select the protocol that is suitable based on the patient data.

In an embodiment, the protocol may be selected by computer 104 based on the aggregate medical information collected by the biometric sensor 102. For example, the biometric sensor 102 and the computer 104 may be used to collected patient data in the northeastern part of Zambia. Patient data collected from members of the Bemba tribe may be tagged with a genetic value “Bemba.” Thereafter, when a new patient of the Bemba tribe is being given a medical examination, the aggregate medical information stored on computer 104 may be searched for any patient data tagged with the genetic value “Bemba.” The search results may be then evaluated to identify a common medical diagnosis and a corresponding protocol to test for the common medical diagnosis. The identified protocol may then be used to test the new patient for the common medical diagnosis since members of the Bemba tribe are prone to that common medical diagnosis.

In Step 216, optionally, patient data is collected based at least on the protocol identified in Step 214. Collecting patient data based on a protocol is described above with relation to Step 206. If Step 206 was performed (e.g., if a first set of patient data was already collected), additional patient data may be collected in Step 216 during a same medical examination as Step 206. For example, patient data may be collected and then immediately compared to the aggregate medical information. Based on an evaluation of the patient data in view of the aggregate medical information, symptoms within the patient data may be identified as one or more indications of a common medical diagnosis found in the aggregate medical information. The protocol determined in Step 214 then may be used to collect additional patient data in Step 216 within the same patient visit. A same medical examination may refer to one or more examinations performed during the same visit by the patient to the hospital, medical office, or other location. A same medical examination may refer to one or more examinations performed during the same visit by an operator to the patient's home, office, etc.

In Step 218, patient data (for example collected in Step 206 or Step 216) may be evaluated in view of a patient community to which that patient belongs. A confidence measure or score of a medical diagnosis for a patient may be computed based on a frequency of a particular medical condition for other patients within a same patient community. The confidence measure or score may indicate a likelihood or probability that the patient has a particular medical condition.

For example, patient data may be collected from a particular patient of Mexican descent using an ultrasound probe to test for Gallstone disease. The patient data may be compared to test results for patients without Gallstone disease and to test results for patients known to have Gallstone disease. Based on the comparison, the particular patient may be diagnosed with a seventy percent likelihood of having Gallstone disease. However, this diagnosis may be modified based on aggregate medical information associated with a patient community of Mexican patients. The aggregate medical information may indicate that the patient community has a high occurrence of Gallstone disease. Based on the high occurrence of Gallstone disease in Mexican patients, the likelihood of the particular patient having Gallstone disease may be increased to eighty percent. In an embodiment, the likelihood of a particular patient having a particular medical condition may be computed based on a percentage or other measure of patients within a same community being diagnosed with the same particular medical condition.

In an embodiment, the score modification may be based on how common the medical diagnosis is within multiple patient communities to which a particular patient belongs. For example, a percentage of patients, suffering from a particular medical condition, of each patient community to which the patient belongs may be identified. An average of the different percentages may be computed and then used to modify the confidence measure of the particular patient suffering from the particular medical condition.

In an embodiment, evaluating patient data may include generating a geographical map identifying locations at which one or more portions of aggregate medical information. The geographical map may further identify locations affected by environmental factors. For example, locations at which patient data for patients diagnosed with asbestosis was collected may be concurrently mapped with locations at which asbestos based products are manufactured. A geographical map may then be used to analyze the relationship between environmental conditions (e.g., asbestos exposure) and patient conditions (e.g., asbestosis).

In an embodiment, the aggregate medical information may be used independently to identify protocols for examining patients. In an embodiment, the aggregate medical information may be used in combination with patient data to identify a protocol to collect additional patient data.

In an embodiment, determining a medical diagnosis based on aggregate medical information may allow for efficient and effective testing of patients within a community. The effect of environmental factors on patients within a community may be easily determined as aggregate medical information may be useful for identifying trends.

In an embodiment, evaluating patient information immediately upon collection in view of aggregate medical information may allow for quickly determining follow-up exams that should be performed on a patient. The quick identification of follow-up exams may allow for completion of follow-up exams within a same medical examination session as an initial exam.

In an embodiment, using an attribute associated with patient data to select aggregate medical information may allow for a computer to select the tests, based on the aggregate medical information, that should be performed on a patient.

In an embodiment, using an attribute associated with patient data to select aggregate medical information associated with that location may simplify identification of conditions from which a patient may be suffering.

In an embodiment, location-based protocols for performing medical examinations may be used for automatically and efficiently updating portable devices that are used for the medical examinations.

### Hardware Overview

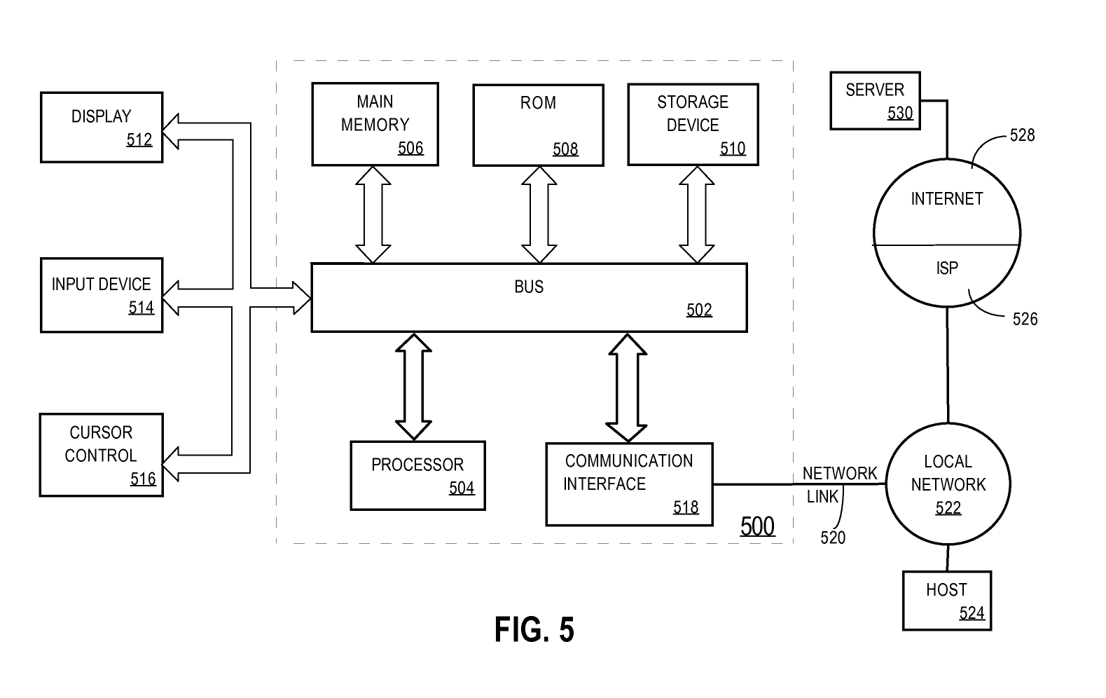

FIG. 5 is a block diagram that illustrates a computer system 500 upon which an embodiment may be implemented. Computer system 500 includes a bus 502 or other communication mechanism for communicating information, and a processor 504 coupled with bus 502 for processing information. Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

The invention is related to the use of computer system 500 for implementing the techniques described herein. According to one embodiment, those techniques are performed by computer system 500 in response to processor 504 executing one or more sequences of one or more instructions contained in main memory 506. Such instructions may be read into main memory 506 from another machine-readable medium, such as storage device 510. Execution of the sequences of instructions contained in main memory 506 causes processor 504 to perform the process steps described herein. In alternative embodiments, hard-wired circuitry may be used in place of or in combination with software instructions to implement the invention. Thus, embodiments are not limited to any specific combination of hardware circuitry and software.

The term “machine-readable medium” as used herein refers to any medium that participates in providing data that causes a machine to operation in a specific fashion. In an embodiment implemented using computer system 500, various machine-readable media are involved, for example, in providing instructions to processor 504 for execution. Such a medium may take many forms, including but not limited to storage media and transmission media. Storage media includes both non-volatile media and volatile media. Non-volatile media includes, for example, optical or magnetic disks, such as storage device 510. Volatile media includes dynamic memory, such as main memory 506. Transmission media includes coaxial cables, copper wire and fiber optics, including the wires that comprise bus 502. Transmission media can also take the form of acoustic or light waves, such as those generated during radio-wave and infra-red data communications. All such media must be tangible to enable the instructions carried by the media to be detected by a physical mechanism that reads the instructions into a machine.

Common forms of machine-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read.

Various forms of machine-readable media may be involved in carrying one or more sequences of one or more instructions to processor 504 for execution. For example, the instructions may initially be carried on a magnetic disk of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are exemplary forms of carrier waves transporting the information.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution. In this manner, computer system 500 may obtain application code in the form of a carrier wave.

In the foregoing specification, embodiments have been described with reference to numerous specific details that may vary from implementation to implementation. Thus, the sole and exclusive indicator of what is the invention, and is intended by the applicants to be the invention, is the set of claims that issue from this application, in the specific form in which such claims issue, including any subsequent correction. Any definitions expressly set forth herein for terms contained in such claims shall govern the meaning of such terms as used in the claims. Hence, no limitation, element, property, feature, advantage or attribute that is not expressly recited in a claim should limit the scope of such claim in any way. The specification and drawings are, accordingly, to be regarded in an illustrative rather than a restrictive sense.

What is claimed is:

1. A computer comprising:
one or more processors;
logic coupled to the one or more processors and comprising one or more stored sequences of instructions which, when executed by one or more processors, cause the one or more processors to perform:
collecting first patient data, associated with at least one attribute, from a patient by one or more biometric sensors based on a first protocol;
evaluating the first patient data based at least on a portion, of aggregate medical information, associated with the at least one attribute;
based on the evaluating, determining a second protocol for collecting additional patient data;
collecting the additional patient data by the one or more biometric sensors based on the second protocol.

2. The computer as recited in claim 1, wherein the instructions for evaluating the first patient data based at least on the portion of the aggregate medical information comprise instructions for:
determining that the first patient data comprises one or more indications of a particular medical diagnosis;
wherein the second protocol tests for the particular medical diagnosis.

3. The computer as recited in claim 1, wherein the instructions for evaluating the first patient data based at least on the portion of the aggregate medical information comprise instructions for:
computing a confidence measure that the first patient data is indicative of a particular medical diagnosis;
wherein the confidence measure is based, at least in part, on a frequency of the particular medical diagnosis in patients associated with the portion of the aggregate medical information.

4. The computer as recited in claim 1, wherein the computer further comprises instructions for:
prior to obtaining the first patient data by the one or more biometric sensors:
obtaining the aggregate medical information by the one or more biometric sensors;
tagging the portion of the aggregate medical information with the at least one attribute.

5. The computer as recited in claim 1, wherein the computer further comprises instructions for:
detecting that the first patient data was collected at a particular location with a geographical area;
determining that the portion of the aggregate medical information was collected within the geographical area;
responsive to the determining that the portion of the aggregate medical information was collected within the geographical area, selecting the portion of the aggregate medical information for evaluating the first patient data.

6. The computer as recited in claim 1, wherein the at least one attribute comprises one or more of:
genetic information of the patient;
an age of the patient;
a nationality of the patient;
an ethnicity of the patient;
a place of residence of the patient;
a place of work of the patient;
a socio-economic group associated with the patient;
a behavioral habit of the patient;
a lifestyle habit of the patient;
a patient condition in which the first patient data was collected;
an environmental condition to which the patient is exposed;
a chemical to which the patient is exposed; or
a pollutant to which the patient is exposed.

7. The computer as recited in claim 1, wherein the computer is communicatively coupled, directly, with the one or more biometric sensors.

8. The computer as recited in claim 1, further comprising instructions for:
generating a geographical map comprising a first plurality of locations at which the portion of the aggregate medical information was collected and a second plurality of locations associated with environmental factors.

9. The computer as recited in claim 1, wherein the first patient data comprises one or more results of an ultrasound probing procedure performed with an ultrasound probe comprising the one or more biometric sensors.

10. The computer as recited in claim 1, wherein the first patient data and the additional patient data are collected by the one or more biometric sensors within a same medical examination session.

11. A computer comprising:
one or more processors;
logic coupled to the one or more processors and comprising one or more stored sequences of instructions which, when executed by one or more processors, cause the one or more processors to perform:
obtaining, from a remote computer, first patient data associated with at least one attribute and collected from a patient by one or more biometric sensors based on a first protocol;
evaluating the first patient data based at least on a portion, of aggregate medical information, associated with the at least one attribute;
based on the evaluating, determining a second protocol for collecting additional patient data;
obtaining, from the remote computer, the additional patient data collected by the one or more biometric sensors based on the second protocol within a same medical examination session as the first patient data.

12. The computer as recited in claim 11, wherein the portion of the aggregate medical information was collected within a particular geographical area and wherein the computer further comprising instructions for:
determining that the remote computer is located within the particular geographical area;
responsive to determining that the remote computer is located within the particular geographical area, selecting the portion of the aggregate medical information collected within the particular geographical area.

13. A computer comprising:
one or more processors;
logic coupled to the one or more processors and comprising one or more stored sequences of instructions which, when executed by one or more processors, cause the one or more processors to perform:
storing patient data collected by one or more biometric sensors as aggregate medical information on the computer;
tagging one or more portions of the aggregate medical information with one or more attributes;
determining that particular patient data, that is to be collected by the one or more biometric sensors, is associated with at least one attribute of the one or more attributes;
responsive to determining that the particular patient data is associated with at least one attribute, selecting a portion of the aggregate medical information that is tagged with the at least one attribute;
determining a common medical diagnosis associated with the portion of the aggregate medical information;
identifying a protocol for testing a patient for the common medical diagnosis;
based on the protocol, collecting the particular patient data from the patient by the one or more biometric sensors.

14. A method comprising:
collecting first patient data, associated with at least one attribute, from a patient by one or more biometric sensors based on a first protocol;
evaluating, by a computer, the first patient data based at least on a portion, of aggregate medical information, associated with the at least one attribute;
based on the evaluating, determining a second protocol for collecting additional patient data;
collecting the additional patient data by the one or more biometric sensors based on the second protocol.

15. The method as recited in claim 14, wherein evaluating the first patient data based at least on the portion of the aggregate medical information comprises:
determining that the first patient data comprises one or more indications of a particular medical diagnosis;
wherein the second protocol tests for the particular medical diagnosis.

16. The method as recited in claim 14, wherein evaluating the first patient data based at least on the portion of the aggregate medical information comprises:
computing a confidence measure that the first patient data is indicative of a particular medical diagnosis;
wherein the confidence measure is based, at least in part, on a frequency of the particular medical diagnosis in patients associated with the portion of the aggregate medical information.

17. The method as recited in claim 14, further comprising:
prior to obtaining the first patient data by the one or more biometric sensors:
obtaining the aggregate medical information by the one or more biometric sensors;
tagging the portion of the aggregate medical information with the at least one attribute.

18. The method as recited in claim 14, further comprising:
detecting that the first patient data was collected at a particular location with a geographical area;
determining that the portion of the aggregate medical information was collected within the geographical area;
responsive to the determining that the portion of the aggregate medical information was collected within the geographical area, selecting the portion of the aggregate medical information for evaluating the first patient data.

19. The method as recited in claim 14, wherein the at least one attribute comprises one or more of:
genetic information of the patient;
an age of the patient;
a nationality of the patient;
an ethnicity of the patient;
a place of residence of the patient;
a place of work of the patient;
a socio-economic group associated with the patient;
a behavioral habit of the patient;
a lifestyle habit of the patient;
a patient condition in which the first patient data was collected;
an environmental condition to which the patient is exposed;
a chemical to which the patient is exposed; or
a pollutant to which the patient is exposed.

20. The method as recited in claim 14, wherein the computer is communicatively coupled, directly, with the one or more biometric sensors.

21. The method as recited in claim 14, further comprising instructions for:
generating a geographical map comprising a first plurality of locations at which the portion of the aggregate medical information was collected and a second plurality of locations associated with environmental factors.

22. The method as recited in claim 14, wherein the first patient data comprises one or more results of an ultrasound probing procedure performed with an ultrasound probe comprising the one or more biometric sensors.

23. The method as recited in claim 14, wherein the first patient data and the additional patient data are collected by the one or more biometric sensors within a same medical examination session.

24. A method comprising:
obtaining, from a remote computer, first patient data associated with at least one attribute and collected from a patient by one or more biometric sensors based on a first protocol;
evaluating the first patient data based at least on a portion, of aggregate medical information, associated with the at least one attribute;
based on the evaluating, determining a second protocol for collecting additional patient data;
obtaining, from the remote computer, the additional patient data collected by the one or more biometric sensors based on the second protocol within a same medical examination session as the first patient data.

25. The method as recited in claim 24, wherein the portion of the aggregate medical information was collected within a particular geographical area and the method further comprising:
determining that the remote computer is located within the particular geographical area;
responsive to determining that the remote computer is located within the particular geographical area, selecting the portion of the aggregate medical information collected within the particular geographical area.

26. A method comprising:
storing patient data collected by one or more biometric sensors as aggregate medical information;
tagging one or more portions of the aggregate medical information with one or more attributes;
determining that particular patient data, that is to be collected by the one or more biometric sensors, is associated with at least one attribute of the one or more attributes;
responsive to determining that the particular patient data is associated with at least one attribute, selecting a portion of the aggregate medical information that is tagged with the at least one attribute;
determining a common medical diagnosis associated with the portion of the aggregate medical information;
identifying a protocol for testing a patient for the common medical diagnosis;
based on the protocol, collecting the particular patient data from the patient by the one or more biometric sensors;
wherein the method is performed by at least one computer.

27. A non-transitory computer readable storage medium comprising instructions, which when executed by one or more processors, cause:
collecting first patient data, associated with at least one attribute, from a patient by one or more biometric sensors based on a first protocol;
evaluating the first patient data based at least on a portion, of aggregate medical information, associated with the at least one attribute;
based on the evaluating, determining a second protocol for collecting additional patient data;
collecting the additional patient data by the one or more biometric sensors based on the second protocol.

28. The computer readable medium as recited in claim 27, wherein the instructions for evaluating the first patient data based at least on the portion of the aggregate medical information comprise instructions for:
determining that the first patient data comprises one or more indications of a particular medical diagnosis;
wherein the second protocol tests for the particular medical diagnosis.

29. The computer readable medium as recited in claim 27, wherein the instructions for evaluating the first patient data based at least on the portion of the aggregate medical information comprise instructions for:
computing a confidence measure that the first patient data is indicative of a particular medical diagnosis;
wherein the confidence measure is based, at least in part, on a frequency of the particular medical diagnosis in patients associated with the portion of the aggregate medical information.

30. The computer readable medium as recited in claim 27, further comprising instructions for:
prior to obtaining the first patient data by the one or more biometric sensors:
obtaining the aggregate medical information by the one or more biometric sensors;
tagging the portion of the aggregate medical information with the at least one attribute.

31. The computer readable medium as recited in claim 27, further comprising instructions for:
detecting that the first patient data was collected at a particular location with a geographical area;
determining that the portion of the aggregate medical information was collected within the geographical area;
responsive to the determining that the portion of the aggregate medical information was collected within the geographical area, selecting the portion of the aggregate medical information for evaluating the first patient data.

32. The computer readable medium as recited in claim 27, wherein the at least one attribute comprises one or more of:
genetic information of the patient;
an age of the patient;
a nationality of the patient;
an ethnicity of the patient;
a place of residence of the patient;
a place of work of the patient;
a socio-economic group associated with the patient;
a behavioral habit of the patient;
a lifestyle habit of the patient;
a patient condition in which the first patient data was collected;
an environmental condition to which the patient is exposed;
a chemical to which the patient is exposed; or
a pollutant to which the patient is exposed.

33. The computer readable medium as recited in claim 27, further comprising instructions for:
generating a geographical map comprising a first plurality of locations at which the portion of the aggregate medical information was collected and a second plurality of locations associated with environmental factors.

34. The computer readable medium as recited in claim 27, wherein the first patient data comprises one or more results of an ultrasound probing procedure performed with an ultrasound probe comprising the one or more biometric sensors.

35. The computer readable medium as recited in claim 27, wherein the first patient data and the additional patient data are collected by the one or more biometric sensors within a same medical examination session.

36. A non-transitory computer readable storage medium comprising instructions, which when executed by one or more processors, cause:
obtaining, from a remote computer, first patient data associated with at least one attribute and collected from a patient by one or more biometric sensors based on a first protocol;
evaluating the first patient data based at least on a portion, of aggregate medical information, associated with the at least one attribute;
based on the evaluating, determining a second protocol for collecting additional patient data;
obtaining, from the remote computer, the additional patient data collected by the one or more biometric sensors based on the second protocol within a same medical examination session as the first patient data.

37. The computer readable medium as recited in claim 36, wherein the portion of the aggregate medical information was collected within a particular geographical area and wherein the computer readable medium further comprising instructions for:
determining that the remote computer is located within the particular geographical area;
responsive to determining that the remote computer is located within the particular geographical area, selecting the portion of the aggregate medical information collected within the particular geographical area.

38. A non-transitory computer readable storage medium comprising instructions, which when executed by one or more processors, cause:
storing patient data collected by one or more biometric sensors as aggregate medical information on the computer;
tagging one or more portions of the aggregate medical information with one or more attributes;
determining that particular patient data, that is to be collected by the one or more biometric sensors, is associated with at least one attribute of the one or more attributes;
responsive to determining that the particular patient data is associated with at least one attribute, selecting a portion of the aggregate medical information that is tagged with the at least one attribute;
determining a common medical diagnosis associated with the portion of the aggregate medical information;
identifying a protocol for testing a patient for the common medical diagnosis;
based on the protocol, collecting the particular patient data from the patient by the one or more biometric sensors.
